(19) United States
(12) Patent Application Publication
(10) Pub. No.: US 2011/0264117 A1
(43) Pub. Date: Oct. 27, 2011

(54) TISSUE JOINING DEVICE AND INSTRUMENT FOR ENABLING USE OF A TISSUE JOINING DEVICE

(75) Inventors: Bradley Edward Layton, Havertown, PA (US); Margaret A. Wheatley, Media, PA (US); Ari Brooks, Cherry Hill, NJ (US); Andrew J. Bohl, Plainsboro, NJ (US); Daniel S. Frame, Salt Lake City, UT (US)

(73) Assignees: Philadelphia Health & Education Corporation d/ba Drexel University College of Medicine, Philadelphia, PA (US); Drexel University, Philadelphia, PA (US)

(21) Appl. No.: 12/990,725

(22) PCT Filed: May 1, 2009
(86) PCT No.: PCT/US09/42528

§ 371 Date: Feb. 16, 2011

Related U.S. Application Data
(60) Provisional application No. 61/049,946, filed on May 2, 2008.

(51) Int. Cl. A61B 17/10; A61B 17/08

(52) U.S. Cl. 606/139; 606/151

(57) ABSTRACT

A tissue joining device and a surgical instrument for employing the tissue joining device are provided. The tissue joining device may be designed such that a first member may be inserted into a second member in discrete increments so that two lumen structures can properly be combined.

### CROSS-REFERENCE TO RELATED APPLICATIONS

This application claims the benefit of U.S. Provisional Application No. 61/049,946 filed May 2, 2008, the entirety of which is incorporated by reference herein.

### FIELD OF THE TECHNOLOGY

The present invention relates to biodegradable tissue joining devices. The present invention also relates to instruments for enabling use of tissue joining devices.

### BACKGROUND

Historically, surgical procedures that require anastomosis of small lumen structures, such as the common bile duct, ureters, and blood vessels have not benefited from the reliability and ease of surgical stapling. Current barriers to the use of surgical staples (or grommets or rivets) in small structures include their size and susceptibility to ill effects from scarring at the tissue joint. When a structure is divided, its blood supply is divided, as well as, making the end very tenuous. This end is then joined to another structure with reduced vascularity. With a thin wall, small lumen size and reduced blood flow, the risk of poor healing, leak or late stricture is very high.

For example, current anastomosis techniques rely on manually suturing the bile duct to the duodenum or employing the use of staple clips to make the connection. However, when these two techniques were compared, complications such as leaks and strictures occurred 18% of the time with clips and 24% of the time with sutures (Castaldo, E. T., M. T. Austin, C. W. Pinson and R. S. Chari (2007a). “Management of the bile duct anastomosis and its complications after liver transplantation.” Transplantation Reviews 21:26-33).

Newer techniques involving laparoscopic reconstruction have been applied to this procedure in recent years, however, a meticulous suturing technique is required and there is no treatment applied in situ to promote healing. Even with use of laparoscopy, the median operative time is around 300 minutes and bile leakage is common. In order to improve the outcome of such surgeries the development of an improved tissue joining device and surgical instrument to employ the tissue joining device which allows surgeons to perform an anastomosis on small anatomical structures with greater ease than conventional suturing is needed.

### SUMMARY

A tissue joining device and a surgical instrument for employing the tissue joining device are provided. The tissue joining device may be designed such that a first member may be inserted into a second member in discrete increments so that two lumen structures can properly be combined.

One example of a tissue joining device according to the present invention may include a first member and a second member. The first member may include a head, a base extending from the head, a plurality of threads extending from a surface of the base, and an aperture extending through the base and the head. The second member may have an aperture extending therethrough, and a plurality of threads extending from a surface of the aperture. The base of the first member may be urged into the aperture of the second member and the threads of the first member may cooperate with the threads of the second member such that the distance that the base passes through the aperture of the second member may be controlled.

The tissue joining device may have several different features to improve its performance. In some embodiment the threads of the first and second members may be buttress shaped. Such a configuration may allow the threads of the first and second members to deflect past each other. For example, in one embodiment each thread may include a bottom surface, a top surface, and a peripheral surface extending up at an angle from the bottom surface and terminating at the top surface. In another embodiment each thread of the first member may include a top surface and an angled surface extending up from a surface of the base and terminating at a distal end of the top surface, while each thread of the second member may include a bottom surface and an angled surface extending down from a surface of the aperture and terminating at a distal end of the bottom surface.

In some embodiments, the threads of each member may define recesses between adjacent threads. For example, in one embodiment a first thread of the second member may be spaced apart from a second thread of the second member such that a first recess may be defined between the first and second threads, and as the base of the first member passes through the aperture of the second member, a first thread of the first member may deflect to pass by the first thread of the second member and may be inserted into the first recess.

In some embodiments, the threads may be rounded. For example a distal end of each thread of the first member may be rounded. Additionally, a distal end of each thread of the second member may be rounded. Such embodiments may ensure that the distal ends of the threads do not become compromised during mating of the two members.

In some embodiments, the base of the first member may include a slit that separates the base into a first leg and a second leg. Such embodiments may allow the two legs to deflect inward when the base of the first member is inserted into the aperture of the second member.

In some embodiments, the first member may be joined to the second member by rotating either the first member or the second member. For example, the base of the first member may include a first portion, a second portion opposite the first portion, a third portion, and a fourth portion opposite the third portion, and the aperture of the second member may include a first portion, a second portion opposite the first portion, a third portion, and a fourth portion opposite the third portion. The first and the third portions of the first member may be void of threads, and the second and fourth portions of the first member may include threads, while the first and third portions of the aperture of the second member may include threads, and the second and fourth portions of the aperture of the second member may be void of threads. Accordingly, the base of the first member may be inserted into the aperture of the second member, and the threads of the two members may engage each other by rotating either the first member or the second member.

An example surgical instrument for joining two tissue portions using a tissue joining device is also provided. In one embodiment, the surgical instrument may include a handle, a body extending from the handle, and a head portion extending from the body. The head portion may have an aperture, and a clip extending through the aperture. The clip may be capable of grabbing a first end of a cable. A second end of the cable may be fastened to a first member of a tissue joining device and the clip may be adapted to translate the first member toward a second member of the tissue joining device to thereby fasten the first and second members together.

The surgical instrument may include several additional features. For example the surgical instrument may also include a knob disposed proximate to the handle and the knob may be in communication with the clip, such that rotation of the knob may translate the first member toward the second member. In some embodiments, the head portion of the surgical instrument may include a recess and the second member may be positioned in the recess. In some embodiments, the surgical instrument may include a button for indicating the amount of force and thus displacement of the first member as it is tightened into the second member. The button may be located proximate to the handle.

The clip of the surgical instrument may include a variety of designs capable of translating the first member of the tissue joining device. For example, in some embodiments the clip may include a first member and a second member attached to the first member. The first member of the clip may include a hook portion and the second member of the clip may be adapted to open and close an opening of the hook portion. When the second member is in an open position, the clip may be able to grab the cable that is fastened to the first member.

The surgical instrument may also include a cutting mechanism. The cutting mechanism may be disposed within the head portion and may be adapted to cut away a portion of the first member of the tissue joining device that extends below the second member of the tissue joining device. In some embodiments, the surgical instrument may include a trigger coupled to the handle, and a cable coupled to the trigger at a first end and to the cutting mechanism at a second end, wherein when the trigger is squeezed the cutting mechanism may cut the portion of the first member that extends below the second member.

A method for joining two tissue portions is also provided. For example the method may include the steps of inserting a first member of a tissue joining device into a first lumen structure, a cord being attached to the first member; inserting a surgical instrument into a second lumen structure, the surgical instrument comprising a head portion having a clip; passing the clip through a wall of the second lumen structure; grabbing the cord with the clip; and translating the first member to securely join the first member with a second member of the tissue joining device.

The method may include several additional steps. For example, in some embodiments the method may include the step of cutting a portion of the first member that extends below the second member. In some embodiments, the method may include the step of suturing the first member to the first lumen structure. In some embodiments, the method may include the step of rotating the first member when a base of the first member is positioned within an aperture of the second member. In some embodiments, the method may include the step of rotating the second member when a base of the first member is positioned within an aperture of the second member.

### BRIEF DESCRIPTION OF THE DRAWINGS

FIG. 1 is a schematic illustrating a tissue joining device joining a small vessel to a larger structure;

FIG. 2 is a schematic illustrating an embodiment of a tissue joining device;

FIG. 3A is a cross-sectional view depicting an embodiment of a first member to be used in a tissue joining device;

FIG. 3B is a bottom view of the first member shown in FIG. 3A;

FIG. 3C is a detailed view depicting threads of the first member shown in FIG. 3A;

FIG. 4A is a top view depicting an embodiment of a second member designed to mate with the first member shown in FIGS. 3A-3C;

FIG. 4B is a cross-sectional view of the second member shown in FIG. 4A;

FIG. 4C is a detailed view depicting threads of the second member shown in FIG. 4A;

FIG. 5A is a cross-sectional view depicting another embodiment of a first member to be used in a tissue joining device;

FIG. 5B is a bottom view of the first member shown in FIG. 5A;

FIG. 6A is a top view depicting an embodiment of a second member designed to mate with the first member shown in FIGS. 5A-5B;

FIG. 6B is a cross-sectional view of the second member shown in FIG. 6A;

FIG. 6C is a detailed view depicting threads of the second member shown in FIG. 6A;

FIG. 7A is side view depicting another embodiment of a first member to be used in a tissue joining device;

FIG. 7B is a cross-sectional view of the first member shown in FIG. 7A;

FIG. 7C is a bottom view of the first member shown in FIG. 7A;

FIG. 7D is a detailed view depicting threads of the first member shown in FIG. 7A;

FIG. 8A is a top view depicting an embodiment of a second member designed to mate with the first member shown in FIGS. 7A-7D;

FIG. 8B is a cross-sectional view of the second member shown in FIG. 8A;

FIG. 8C is a detailed view depicting threads of the second member shown in FIG. 8A;

FIG. 9 is a schematic illustrating another embodiment of a tissue joining device;

FIG. 10 is a side view depicting an embodiment of a surgical instrument device;

FIG. 11 is a detailed view depicting an example adjustment knob of the surgical instrument shown in FIG. 10;

FIG. 12A is a detailed view depicting an example head portion of the surgical instrument shown in FIG. 10;

FIG. 12B is a detailed view depicting an example clip of the head portion shown in FIG. 12A;

FIG. 13A is a detailed view depicting and example cutting mechanism of the surgical instrument shown in FIG. 10;

FIG. 13B is a detailed view of the cutting mechanism shown in FIG. 13A;

FIG. 14A is a cross-sectional view depicting an uncut first member;

FIG. 14B is a cross-sectional view depicting the uncut member of FIG. 14A after it has been cut;

FIG. 15 is a perspective view depicting the head portion of FIG. 12A detached from a body portion; and

FIG. 16 is a schematic illustrating another embodiment of a tissue joining device.

### DETAILED DESCRIPTION OF ILLUSTRATIVE EMBODIMENTS

Provided is a tissue joining device, such as a staple, a rivet or a grommet, for small lumen structures, the device is fabricated with a variable locking mechanism to properly join two lumen structures. The tissue joining device may be made from a polymer, a ceramic and/or a metal. For example, the tissue joining device may be composed of an FDA-approved biocompatible, biodegradable polymer, such as, but not necessarily limited to, polylactic-co-glycolitic acid (PLGA). The ratio of lactide to glycolide may range between 100:0 and 75:25. A ratio with less than 75% lactide may absorb too much water and swell, possibly rendering the tissue joining device mechanically unstable. The tissue joining device may be made of a material tailored to accommodate certain needs such as ease of insertion, maintain a sealed junction between tissues, minimize tissue damage, and/or degrade over a time period equal to the tissue healing rate. For example, the tissue joining device may degrade due to exposure to bodily fluids but at a rate that is commensurate with the rate of healing such that before the device has completely degraded, the healing tissue is capable of supporting physiological loading conditions. The tissue joining device may be designed such that the mechanical properties are optimized such that the traction force between the staple and tissue is of sufficiently great magnitude to maintain mechanical integrity of the joined tissues while being of sufficiently small magnitude such that the tissue is not damaged. Material properties may be optimized based on standard material testing procedures such as ASTM standard D638-03. For example, the material may have a Young's Modulus of 81.51 MPa and a failure stress of 3.71 MPa. The tissue joining device may deliver bioactive agents at a pre-programmed rate to improve healing. For example, the tissue joining device may contain a bioactive drug that may promote stronger and more rapid healing at the point of anastomosis. The bioactive drug may be present in one or more of the tissue joining device subcomponents or it may be present in a bioactive polymeric ring. The tissue joining device may also include a contrast agent such as an iodine compound for X-rays, or a gadolinium compound for MRIs, s that the tissue joining device may be monitored non-invasively. The tissue joining device and its components may be manufactured at a range of sizes proper for the anatomical dimensions of the tissues being joined.

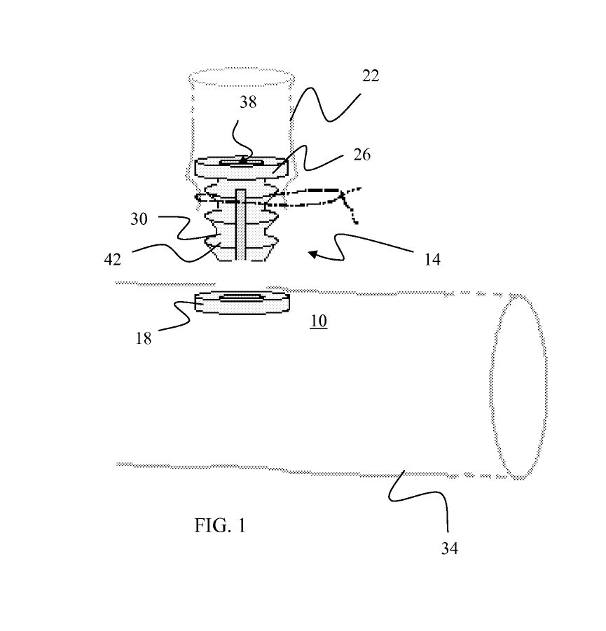

FIG. 1 illustrates the general strategy for joining two lumen structures using a tissue joining device. As shown, a tissue joining device 10 includes a first member 14 and a second member 18. The first member 14 may be sutured to or otherwise connected to a first lumen structure 22, such as a bile duct. The first member 14 may include a head 26, and a base 30 extending from head 26. Base 30 of first member 14 may be inserted through an incision in a second lumen structure 34 such as the small intestine, either manually or mechanically, such as with a specifically designed surgical instrument, wherein second member 18 and first member 14 may mate to thereby connect the first and second lumen structures 22 and 34 together. First member 14 may have a hollow aperture 38 extending through head 26 and base 30 to allow fluid flow through first member 14. To enhance the traction between the lumen structures 22 and 34 and the tissue joining device 10, surface features such as stubs may be added to a bottom surface of head 26 of first member 14 and to a top surface of second member 18. The stubs may have a height of approximately 100 micrometers to 1 mm.

The first member 14 may mate with second member 18 and may allow for variation in the compression of the lumens being joined. After the surgical procedure, tissue joining device 10 may degrade into surrounding interstitial fluids as the lumen structures heal. To promote healing, a drug such as TGF-β1 may be released as tissue joining device 10 degrades.

Tissue joining device 10 may include a series of threads 42 circulating the outside diameter of base 30, as well as a series of threads (not shown) circulating the inside diameter of second member 18. The two sets of threads may act as the fastening mechanism of tissue joining device 10. The threads may extend either in a screw like manner or laterally depending on the embodiment. In the embodiment shown, force applied to first member 14 preferably is great enough so that threads 42 may deflect enough to allow them to pass by the threads of second member 18. The force preferably is not so great, however, that the threads break off due to shear forces.

Once the threads have been interlocked, first and second members 14 and 18 preferably do not come apart in the presence of biological processes or everyday post-operation patient activity. As stated earlier, first member 14 may have multiple threads 42. The number of threads 42 and axial distance between threads may be sufficient to allow for variability in the displacement between second member 18 and first member 14 as the connection is being made. Typical axial (pitch) distances between threads 42 may vary but preferably will not be less than ten micrometers and not more than one millimeter. Such a feature may allow a surgeon to adjust the tightness of the junction between the two lumen structures ensuring a secure connection without causing tissue necrosis from excess pressure. This adjustability may also allow the surgeon to vary the junction for anatomical ranges of tissue thickness of the two structures 22 and 34 to be joined. Once the first and second members 14 and 18 are joined, excess of first member 14 may be cut away.

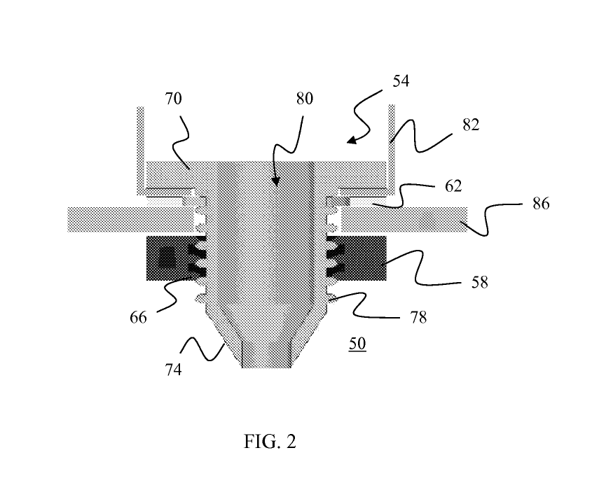

FIG. 2 is a cross-sectional view illustrating a tissue joining device having a drug eluting ring. As shown, a tissue joining device 50 includes a first member 54, a second member 58, and a biodegradable drug eluting ring 62. The second member 58 may be a collar and may include internal threads 66. The first member 54 may include a head 70 and a base 74 having external threads 78. The first member 54 may also include a hollow aperture 80 extending through the head 70 and base 74. When second member 58 and first member 54 mate, a first lumen structure 82 may be joined to a second lumen structure 86. To be sure that lumen structures 82 and 86 are securely joined, threads 78 of first member 54 engage threads 66 of second member 58 when the two members have been mated.

As shown, drug eluting ring 62 may be placed between first member 54 and second member 58. While first member 54 and second member 58 mechanically join lumen structures 82 and 86, drug eluting ring 62 may provide drug delivery directly to the site of the anastomosis and to nearby tissue. As ring 62 degrades, drugs may continuously elude thereby promoting tissue growth as first member 54 and second member 58 degrade in the body. If the drug is an antibiotic, infection may be prevented for the duration of release. If the drug is a chemotherapeutic, residual malignancy may be treated in the local.

In order to achieve maximum efficacy, the drug release rate by drug ring 62 may be sustained over a period sufficient to significantly enhance the healing rate and thus recovery. This elution period has previously been estimated to be a minimum of two weeks after surgery. As a conservative estimate, tissue joining device 50 preferably should degrade in no less than four weeks, as this is the upper limit for the healing time associated with this type of surgery.

As stated earlier, the drug used may be protein TGF-β1. Since the denaturation temperature of TGF-β1 is expected to be significantly less than the melting temperatures required for forming the tissue joining device components by for example injection molding, ring 62 preferably is made by a method such as compression molding or solvent evaporation. The expected weight percentage of drug to ring material may be between 1% to 5%, however, this percentage and drug type may be varied beyond these limits depending on the surgical application. An inert, such as a sugar-like lactose may be added to enhance the drug delivery profile.

If the first lumen structure 82 is a bile duct and the second lumen structure 86 is a small intestine, tissue joining device 50 may be manufactured for such a use. For example, in this case, the outer diameter of second member 58 may be between 4 and 12 mm, as this is the range of diameters for the human bile duct. Because of the relative fragility of the bile duct, first member 54 should be smaller in diameter than the bile duct in order to reduce stress on the tissue. The inner diameter (hollow aperture 80) of first member 54 preferably should be as large as possible in order to allow for the unrestricted flow of bile. However, first member 54 preferably is thick enough to withstand the mechanical forces applied during mating with second member 58.

The first member 54 and the second member 58 will preferably be designed to have threads 66 and 78 oriented so that first member 54 can snap into second member 58 and allow for variation for tightening. For example, the threads may have a variation between 0.7 mm and 1.2 mm. Accordingly, second member 58 may have an inner diameter which may allow for overlap between threads 66 and 78 of first member 54 and second member 58 respectively. The inner diameter of second member 58 preferably is selected such that it is small enough that there is enough overlap between the components that they do not come apart and large enough that there are not excessive shear forces caused by sliding between the components. Thus, when the two parts are put together, threads 66 and 78 should not shear off.

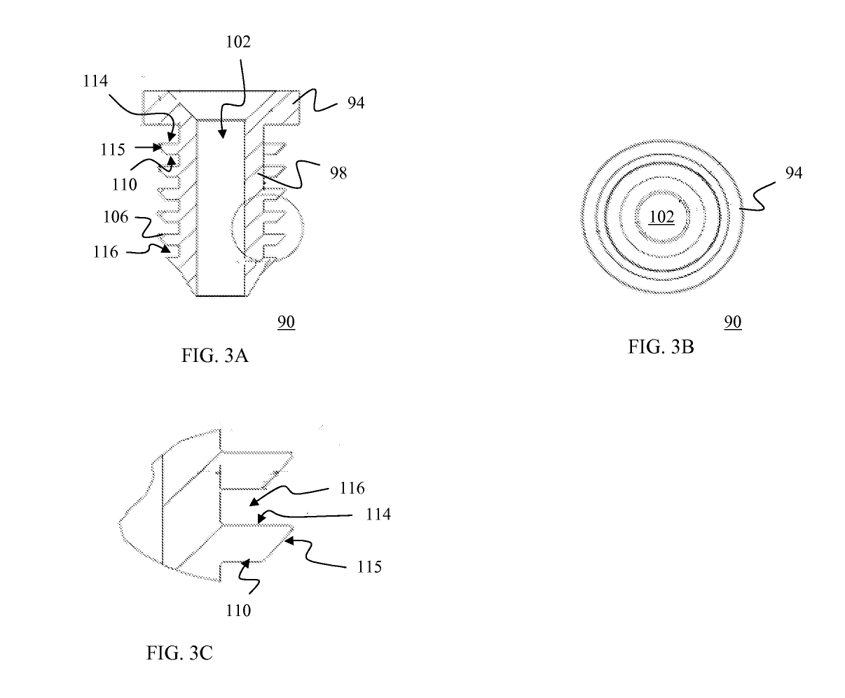

The tissue joining device preferably is designed such that the first member may slide into the second member in discrete intervals. There are a number of ways to achieve this effect. For example, FIGS. 3A-9 depict several different features that the tissue joining device may have.

FIGS. 3A-3C depict a first member of a tissue joining device having buttress shaped threads. As shown in FIGS. 3A and 3B, a first member 90 may include a head 94, a base 98 and an aperture 102 extending therethrough. As shown in FIGS. 3A and 3C, base 98 may include several threads 106 extending from an external surface. Each thread 106 may be buttress shaped having a bottom surface 110, a top surface 114, and a peripheral surface 115 that extends up from bottom surface 110 at an angle and terminates at top surface 114. Accordingly, top surface 114 may be longer than the bottom surface 110 for each thread 106. Additionally, each thread 106 may be spaced apart from an adjacent thread 106 such that a recess 116 may be formed therebetween. Because of the buttress shape to threads 106, the insertion force required to insert first member 90 into a second member may be less than the extraction force required for removal.

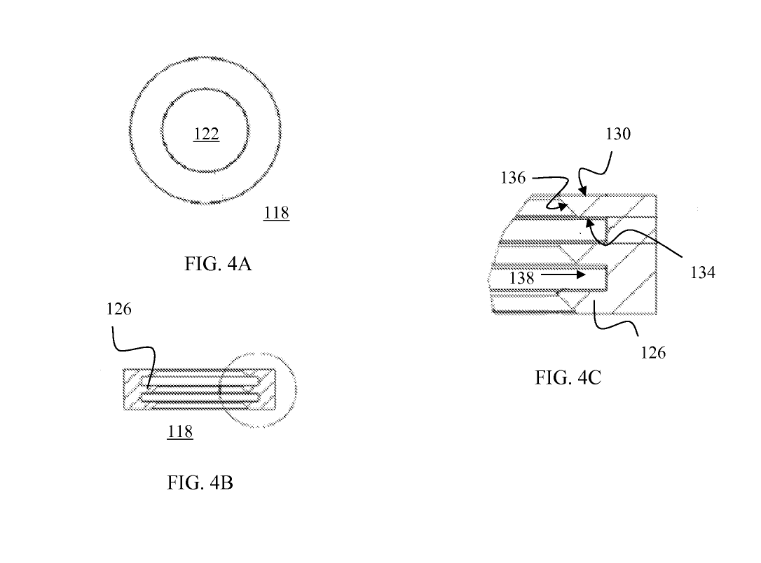

FIGS. 4A-4C depict a corresponding second member 118 to first member 90. As shown in FIG. 4A, second member 118 may be a collar and may include an aperture 122 extending therethrough. As shown in FIGS. 4B and 4C, second member 118 may include internal threads 126. As shown, threads 126 may also be buttress shaped having a top surface 130, a bottom surface 134, and a peripheral surface 136 extending at an angle from bottom surface 134 to top surface 130. Similar to first member 90, each thread 126 may be spaced apart from an adjacent thread 126 such that a recess 138 may be formed therebetween.

The first member 90 and the second member 118 are designed to mate with each other. For example, when first member 90 mates with second member 118, threads 106 of first member 90 may incrementally be inserted into a corresponding recess 138 of second member 118, and threads 126 of second member 118 may incrementally be inserted into a corresponding recess 116 of first member 90. Because peripheral surfaces 115 and 136 extend up at angles, the insertion force may be less than the extraction force.

To ensure that the desired effects of the tissue joining device are achieved, first member 90 and second member 118 may have specific dimensions. For example, recesses 138 may have a height that is greater than the height of threads 106 of first member 90. Furthermore, top surfaces 130 of threads 126 may be longer than top surfaces 114 of threads 106, and bottom surfaces 134 of threads 126 may be shorter than bottom surfaces 110 of the threads 106. Such dimensioning may ensure that threads 106 and 126 do not shear off during mating of the two components, and may also ensure that first member 90 stays fastened to second member 118 once mated.

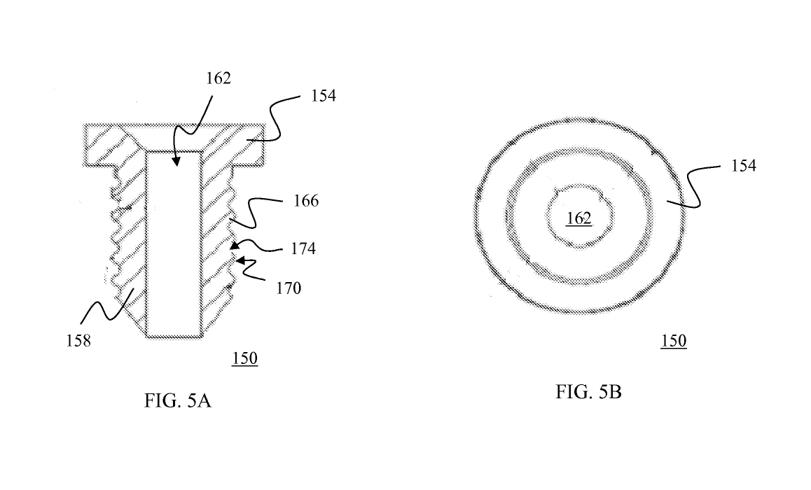

FIGS. 5A and 5B depict a first member of a tissue joining device having rounded thread tips. Such a configuration may ensure that the thread tips do not become compromised during insertion. As shown in FIG. 5A, a first member 150 may include a head 154, a base 158, and an aperture 162 extending therethrough. As shown in FIG. 5A, base 158 may include several threads 166 extending from an external surface. Each thread 166 may be rounded at a distal end 170. Additionally, each thread 166 may be spaced apart from an adjacent thread 166 such that a recess 174 may be formed therebetween. Each recess 174 may also be rounded. Because of the rounded ends 170, stress at the thread tips may be reduced during insertion of first member 150 into a second member.

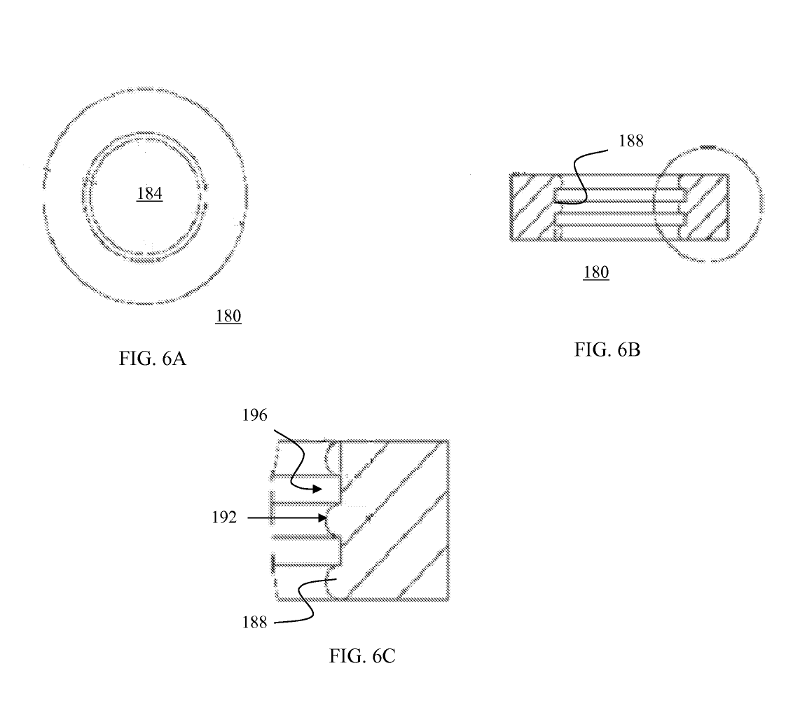

FIGS. 6A-6C depict a corresponding second member 180 to first member 150. As shown in FIG. 6A, second member 180 may be a collar and may include an aperture 184 extending therethrough. As shown in FIGS. 6B and 6C, second member 180 may include internal threads 188. As shown, threads 188 may also be rounded at a distal end 192. Similar to first member 150, each thread 188 may be spaced apart from an adjacent thread 188 such that a recess 196 may be formed therebetween.

The first member 150 and the second member 180 are designed to mate with each other. For example, when first member 150 mates with second member 180, threads 166 of first member 150 may incrementally be inserted into a corresponding recess 196 of second member 180, and threads 188 of second member 180 may incrementally be inserted into a corresponding recess 174 of first member 150. Because of the rounded distal ends 170 and 192, stresses acting on the thread tips may be reduced during mating of the members.

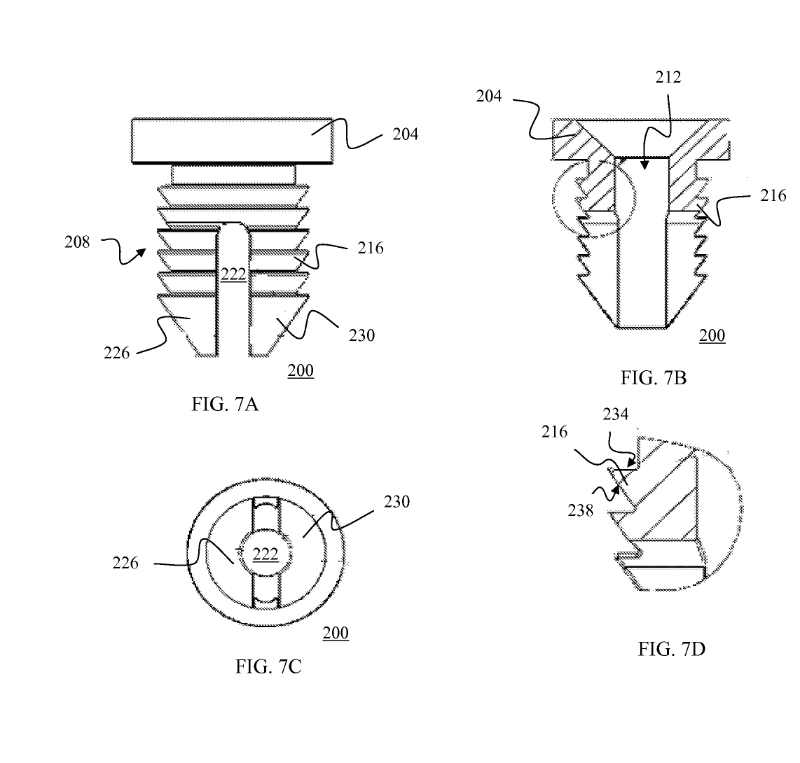

FIGS. 7A-7D depict a first member of a tissue joining device having a stress relieving slit cut in the side of the base. As shown in FIGS. 7A-7C, a first member 200 may include a head 204, a base 208 and an aperture 212 extending therethrough. As shown, the base 208 may include several threads 216 extending from an external surface, and a slit 222 separating base 208 into a first leg 226 and a second leg 230. Each thread 216 may include a top surface 234 and an angled surface 238 that extends up from the external surface of base 208 and terminates at a distal end of top surface 234. Slit 222 preferably is long enough to allow for bending to occur but not so long as to allow leakages post-surgery. For example, slit 222 may extend up three quarters of base 208. First leg 226 and second leg 230 may be adapted to deflect toward each other during surgical insertion.

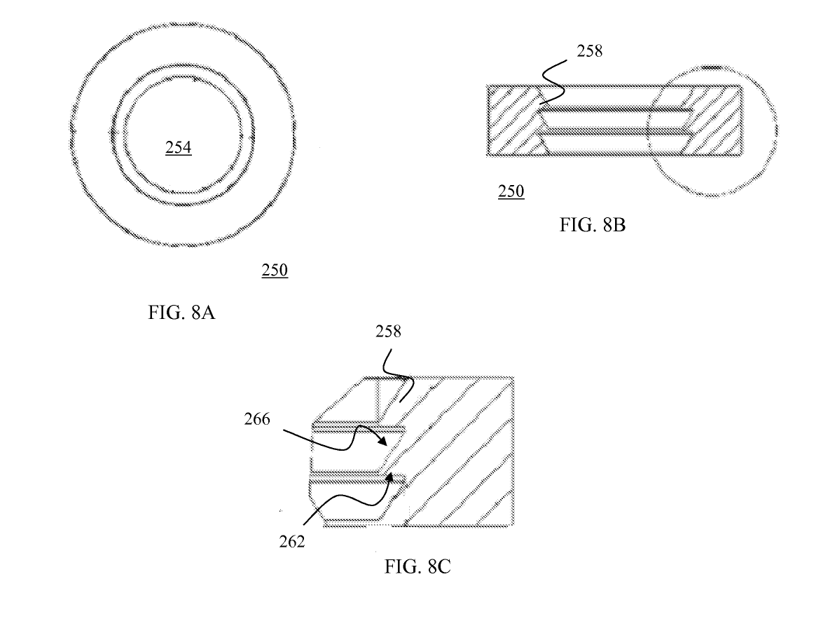

FIGS. 8A-6C depict a corresponding second member 250 to first member 200. As shown in FIG. 8A, second member 250 may be a collar and may include an aperture 254 extending therethrough. As shown in FIGS. 8B and 8C, second member 250 may include internal threads 258 extending from a surface of aperture 254. As shown, threads 258 may each include a bottom surface 262 and an angled surface 266 that extends down from the surface of aperture 254 of second member 250 and terminates at bottom surface 262.

The first member 200 and the second member 250 are designed to mate with each other. For example, when first member 200 mates with second member 250, threads 216 of first member 200 may incrementally mate with a corresponding thread 258 of second member 250. During insertion of base 208 into aperture 254, first and second legs 226 and 230 may deflect toward each other thereby reducing shear stress on the threads. When threads 216 are aligned with the corresponding threads 258 the first and second legs 226 and 230 may deflect back to their original position.

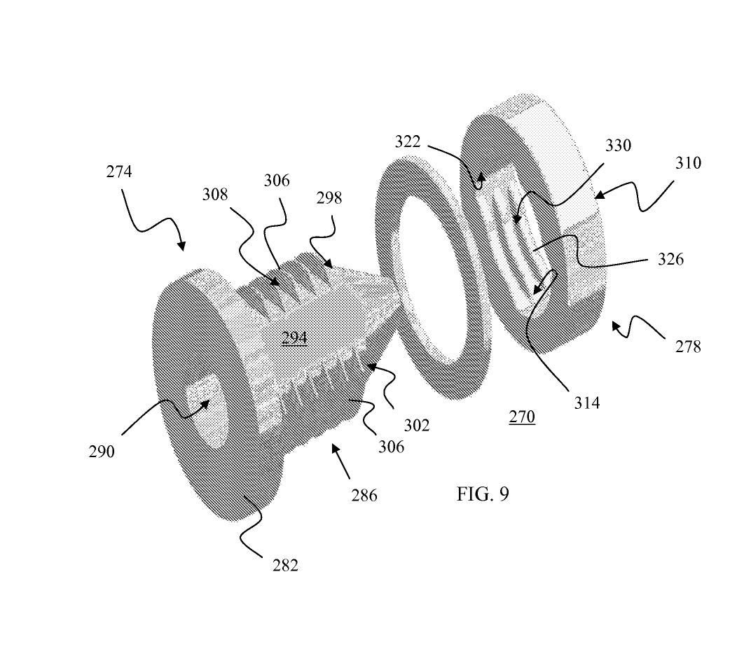

FIG. 9 depicts a tissue joining device that requires rotation for connection to be made between the first and second members. As shown, a tissue joining device 270 may include a first member 274 and a second member 278. The first member 274 may include a head 282, a base 286, and an aperture 290 extending therethrough. The base 286 may include a first portion 294, a second portion (not shown) opposite the first portion 294, a third portion 298, and a fourth portion 302 opposite the third portion 298. As shown, the third and fourth portions 298 and 302 may include threads 306, while the first 294 and second portions may be void of threads. Threads 306 may be spaced apart from each other such that a recess 308 is formed between adjacent threads 306.

As shown, the second member 278 may be a collar and may include an aperture 310 extending therethrough. Aperture 310 may include a first portion 314, a second portion (not shown) opposite the first portion 314, a third portion 322, and a fourth portion (not shown) opposite the third portion 322. As shown, the first 314 and second portions may include threads 326, and the third 322 and fourth portions may be void of threads. Threads 326 may be spaced apart from each other such that a recess 330 is formed between adjacent threads 326.

The first member 274 and the second member 278 are designed to mate with each other. To do so, base 286 should be oriented such that during insertion into aperture 310 of second member 278, threads 306 of base 286 should align with the portions of aperture 310 that are void of threads. Once first member 274 is in a desired position, first member 274 may be rotated (for example 90 degrees) such that threads 306 engage a corresponding recess 330 of the second member 278 and threads 326 engage a corresponding recess 308 of first member 274 to thereby fasten the two members together. It should be understood that either member may be rotated.

A surgical instrument specifically designed to allow for the effective union of lumen structures is also provided. Such an instrument may be capable of clamping two members, such as a second member and a first member of the previously described tissue joining devices, together. The instrument may apply a force and displacement sufficient to effectively join the two structures such that post-surgery leakage and mechanical failure may be minimized, but may not exceed a displacement limit that could cause ischemia or necrosis in the joined tissues.

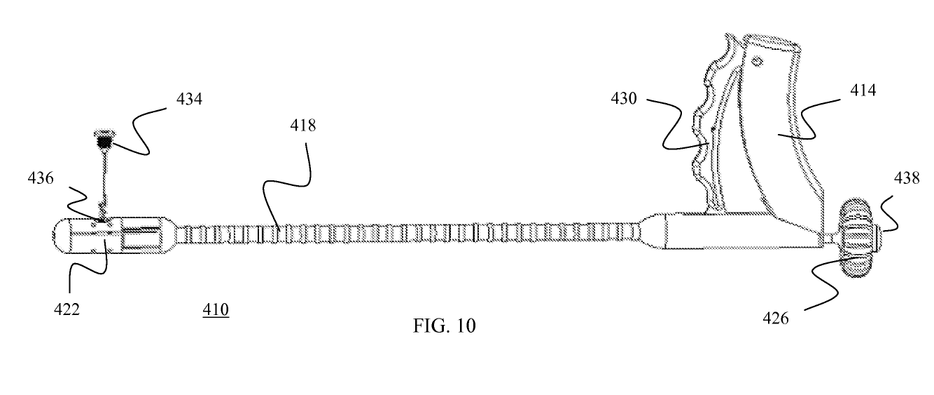

FIG. 10 depicts an example surgical instrument 410 capable of joining two lumen structures effectively. As shown, surgical instrument 410 may include a handle 414, a body 418, and a head portion 422.

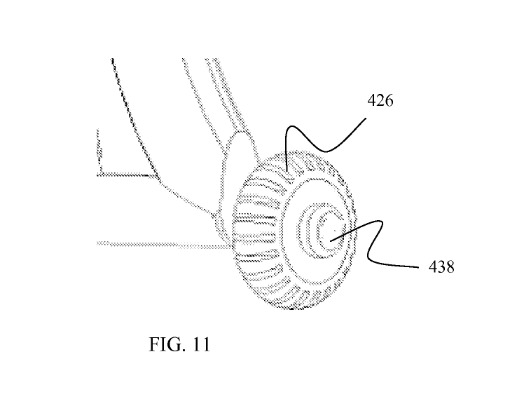

Handle 414 preferably will remain outside of the body so that a surgeon may maneuver and operate the instrument 410 with ease. Handle 414 may include an adjustment knob 426 and a trigger 430. The adjustment knob 426 may be positioned on a backside of handle 414 and may be used to adjust the distance between a first member 434 and a second member 436 of a tissue joining device. As shown in FIG. 11, adjustment knob 426 may include a button 438 that may be spring loaded. The button 438 may indicate the amount of force and thus displacement of first member 434 as it is tightened into the anastomosis site. Once first member 434 is set in place, trigger 430 may be used to cut away an excess portion of first member 434.

Referring back to FIG. 10, the body 418 may extend from the handle 414 and may span a distance from the surgeon's hand to a point of operation such as an area of the small intestine in either an open or laparoscopic procedure. The body 418 preferably is flexible, and thus may be made of a flexible material, such as rubber. Alternatively, body 418 may have a flexible design such as a multiple ball and socket joint design. Through the use of rubber or multiple ball and socket joints, body 418 may be capable of withstanding longitudinal tension and compression and yet be flexible enough to navigate through a lumen.

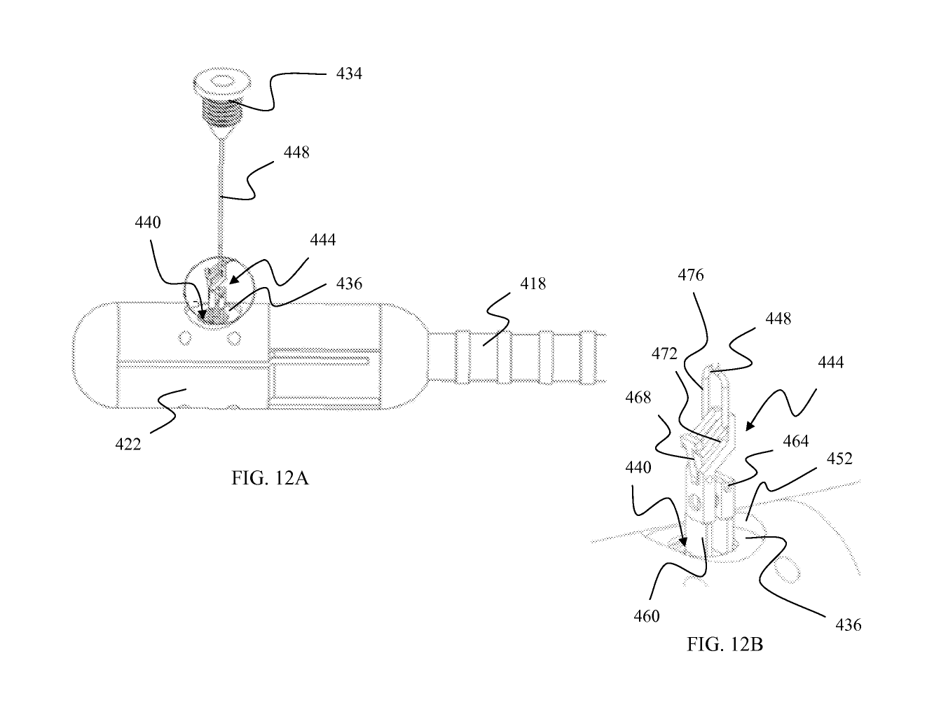

As shown in FIGS. 10 and 12A, the head portion 422 may extend from a distal end of the body 418. The head portion 422 may be made of a material such as polycarbonate or polypropylene. As shown, the head portion 422 may include an aperture 440, a clip 444, and a cable 448. The aperture 440 may include a recess 452 for holding the second member 436. As shown in FIG. 12B, the clip 444 may extend from the aperture 440 and may be adapted to clip onto the cable 448. For example, in the embodiment shown, the clip 444 includes a first portion 460, a second portion 464, and a third portion 468. The first portion 460 includes a hook 472 adapted to hold onto cable 448. The second portion 464 may be coupled to both the third portion 468 and button 438 of handle 414. When button 438 is employed, second portion 464 may adjust and move third portion 468 away from first portion 460 to thereby provide access to hook 472. Once button 438 is released, third portion 468 will move back to its original position thereby closing clip 444. As shown in FIGS. 12A and 12B, cable 448 may be attached to first member 434 at one end, and may include a loop 476 at the other end capable of being hooked onto clip 444.

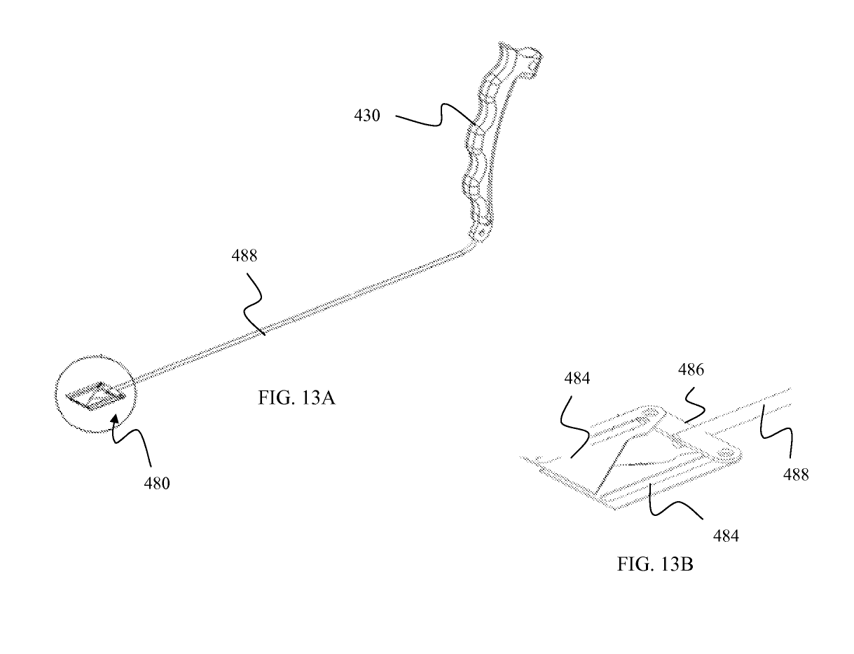

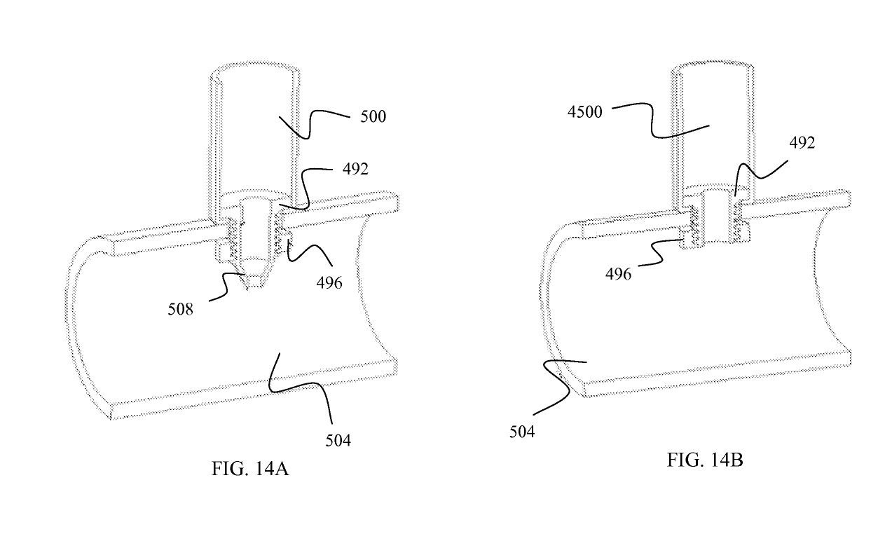

As shown in FIG. 13A the surgical instrument may also include a cutting mechanism 480 adapted to cut away an excess portion of a first member. As shown in FIG. 13B, cutting mechanism 480 may include two separate blades 484 rotatably coupled to a support member 486. Referring back to FIG. 13A, a cable 488 may extend from cutting mechanism 480 to trigger 430, such that when trigger 430 is squeezed, blades 484 will pull across a portion of the first member, just under the second member, shearing it from the rest of the first member, which is to remain. The sheared off portion of the first member may fall into head portion 422 for removal or be passed with the feces. For example, FIG. 14A depicts a first member 492 and a second member 496 joining a first lumen structure 500 to a second lumen structure 504. As shown, when first member 492 is fastened to second member 496, a portion 508 of the first member 492 extends below second member 496. As shown in FIG. 14B, portion 508 may be removed to allow for adequate flow within second lumen structure 504.

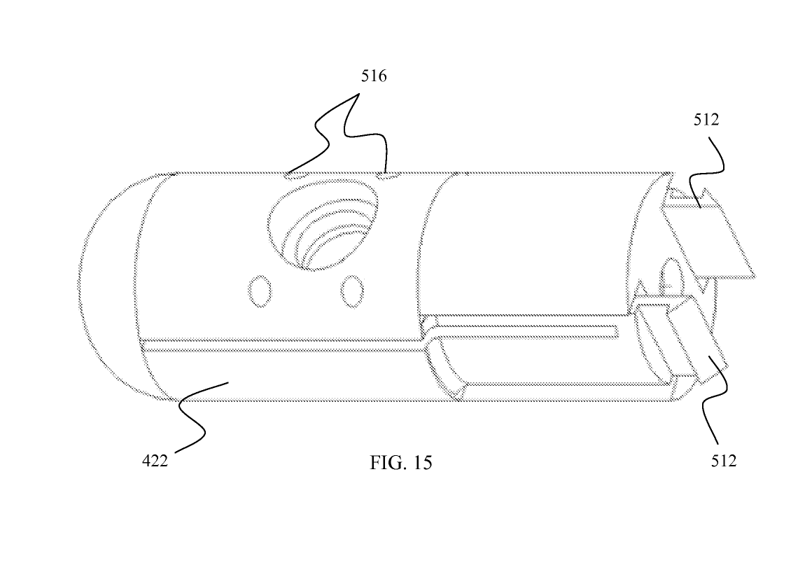

As shown in FIG. 15, head portion 422 may be detachable from the body. In the embodiment shown, head portion 422 includes clips 512 extending from a back side. Clips 512 may enable head portion 422 to be clipped to and detached from the body. When head portion 422 is detached, cable 488 may be released from trigger 430 so that the whole unit can be disposed of after surgery. Head portion 422 may be compatible with common endoscopes. Therefore, head portion 422 and all of its components and functions may be operated using the working channels of a common endoscope.

As shown in FIG. 15, head portion 422 may also include holes 516 extending into head portion 422. Guide pins (not shown) may be inserted into holes 516 to align the cutting mechanism 480 within head portion 422.

In operation first member 434 along with cord 448 may be inserted into a first lumen structure, such as the bile duct of a patient, and purse-string sutured into place to prevent first member 434 from detaching from the first lumen structure. The surgical instrument 410 may then be inserted into a second lumen structure, such as the small intestine, either through the esophagus or laproscopically into the gut until it reaches the point of anastomosis. Once instrument 410 is in position, a small incision may be made to allow clip 444 to pass through the small intestine wall. Next, clip 444 may be employed by using the adjustment knob until a sufficient length is obtained for clip 444 to grab cord loop 476.

The surgeon may now use adjustment knob 426 to form the anastomotic joint. The adjustment knob 426 may be comprised of a knob, a shaft with an external thread and a slotted adapter with an internal thread that may be attached to clip 444. As adjustment knob 426 is rotated, the adapter may translate to pull first member 434 into place within second member 436 until a suitable junction between the first and second lumens is obtained. A junction that is too loose may lead to a joint susceptible to leakage and a junction that is too tight may lead to ischemia or necrosis of the site. Therefore, a combination of the surgeon's attentiveness to the force indicator on button 438 and force feedback received by the surgeon through the torque of adjustment knob 426 may be required for a successful procedure.

The instrument 410 may connect first member 434 to second member 436 a number of different ways. For example, in one embodiment, instrument 410 may pull first member 434 through second member 436, such that mechanical interference between the to members through a combination of geometry and material compliance provides for sufficient binding force to maintain attachment under normal physiological loading conditions. In another embodiment, clip 444 may be capable of sustaining torsion sufficient to allow first member 434 and second member 436 to rotate axially relative to each other such that a secure mechanical connection is made.

Once anastomosis is complete, a protruding portion of first member 434 not in contact with second member 436 may be removed to allow for adequate flow within the second lumen structure (for example, the small intestine). This may be accomplished by using a cutting mechanism, such as cutting mechanism 480 or an abrasive cable drawn either in a reciprocating or continuous manner along the surface of the protruding portion of first member 434. The sheared-off portion of first member 434 may fall into head portion 422 and second member 436 may be pulled out of recess 452 of head portion 422. The sheared-off portion and cord 448 may remain securely inside head portion 422 to facilitate removal without leaving any unwanted foreign objects in the patient. Once the procedure is complete, the surgeon may withdraw instrument 410 and close all incisions.

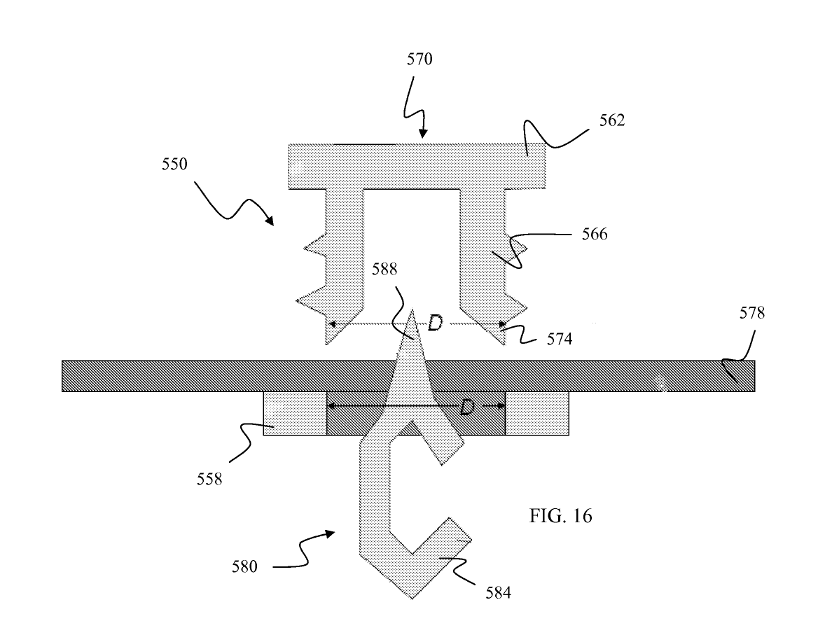

FIG. 16 schematically depicts a tissue joining device having a cutting feature for cutting through a tissue, such as a lumen. As shown, a tissue joining device 550 may include a first member 554 and a second member 558. The first member 554 may include a head 562, a base 566, and an aperture 570 extending therethrough. The base 566 may include a sharp cutting edge 574 that may cut through a tissue 578, as base 566 passes through second member 558. Preferably, sharp cutting edge 574 has a diameter that is approximately equal to an inner diameter of second member 558. It should be appreciated that a top edge of second member 558 may also include a sharp cutting edge.

Additionally, FIG. 16 schematically depicts a clip having a penetration feature for cutting through a tissue, such as a lumen. As shown, a clip 580 may include a hook 584, and a cutting feature 588. In operation, clip 580 may extend through second member 558 and penetrate tissue 578 with the cutting feature 588. The clip 580 may then grab a cable that is connected to first member 554 with hook 584. Hook 584 may be guided with an insert designed for the task, and equipped with a device to visualize the procedure. The guide may be an instrument (having a diameter between 2 mm and 10 mm) that is rigid, straight, malleable, or steerable to conform with requirements of the procedure. The guide may be used in open or laparoscopic/endoscopic surgery. The guide may have at its tip an aperture to rasp and guide the first member into the locking mechanism on the head portion of the tissue joining device. A modification to the head portion and/or the guide may provide guidance by any wavelength of light, radiofrequency, or magnetic means.

The foregoing description is provided for the purpose of explanation and is not to be construed as limiting the invention. While the invention has been described with reference to preferred embodiments or preferred methods, it is understood that the words which have been used herein are words of description and illustration, rather than words of limitation. Furthermore, although the invention has been described herein with reference to particular structure, methods, and embodiments, the invention is not intended to be limited to the particulars disclosed herein, as the invention extends to all structures, methods and uses that are within the scope of the appended claims. Those skilled in the relevant art, having the benefit of the teachings of this specification, may effect numerous modifications to the invention as described herein, and changes can be made without departing from the scope and spirit of the invention as defined by the appended claims. Furthermore, any features of one described embodiment can be applicable to the other embodiments described herein.

What is claimed:

1. A device for joining tissue, the device comprising:
a first member having a head, a base extending from the head, a plurality of threads extending from a surface of the base, and an aperture extending through the base and the head; and
a second member having an aperture extending therethrough, and a plurality of threads extending from a surface of the aperture,
wherein (i) the base of the first member is urged into the aperture of the second member, and (ii) the threads of the first member cooperate with the threads of the second member such that the distance that the base passes through the aperture of the second member is controlled.

2. The device of claim 1, wherein the threads of the first member are buttress shaped.

3. The device of claim 2, wherein each thread of the first member includes a bottom surface, a top surface, and a peripheral surface extending up at an angle from the bottom surface to the top surface.

4. The device of claim 2, wherein each thread of the first member includes a top surface and an angled surface extending up from the surface of the base and terminating at a distal end of the top surface.

5. The device of claim 1, wherein the threads of the second member are buttress shaped.

6. The device of claim 5, wherein each thread of the second member includes a bottom surface, a top surface, and a peripheral surface extending up at an angle from the bottom surface to the top surface.

7. The device of claim 5, wherein each thread of the second member includes a bottom surface and an angled surface extending down from the surface of the aperture and terminating at a distal end of the bottom surface.

8. The device of claim 1, wherein (i) a first thread of the second member is spaced apart from a second thread of the second member such that a first recess is defined between the first and second threads, and (ii) as the base passes through the aperture of the second member a first thread of the first member deflects to pass by the first thread of the second member and is inserted into the first recess.

9. The device of claim 1, wherein a distal end of each thread of the first member is rounded.

10. The device of claim 1, wherein a distal end of each thread of the second member is rounded.

11. The device of claim 1, wherein the base includes a slit that separates the base into a first leg and a second leg, and each leg is adapted to deflect when the base is inserted into the aperture of the second member.

12. The device of claim 1, wherein (i) the base includes a first portion, a second portion opposite the first portion, a third portion, and a fourth portion opposite the third portion, (ii) the aperture of the second member includes a first portion, a second portion opposite the first portion, a third portion, and a fourth portion opposite the third portion, (iii) the first and the third portions of the first member are void of threads, and the second and fourth portions of the first member include threads, and (iv) the first and third portions of the aperture of the second member include threads, and the second and fourth portions of the aperture of the second member are void of threads.

13. The device of claim 1, wherein the second member defines a collar.

14. The device of claim 1, further comprising a drug eluting ring.

15. The device of claim 1, wherein the base further comprises a sharp cutting edge.

16. The device of claim 1, wherein the first member is made of a material having mechanical properties that are optimized such that the traction force between the first member and the tissue maintains mechanical integrity of the joined tissues and does not damage the tissue.

17. The device of claim 1, wherein at least one of the first and second members is made of a material that degrades due to exposure to bodily fluids.

18. The device of claim 17, wherein the material degrades at a rate that is commensurate with the rate of healing.

19. The device of claim 1, wherein at least one of the first and second members comprises a pharmaceutically active ingredient.

20. A method of joining two portions of tissue, at least one portion being tubular, the method comprising:
(a) securing a first member in the tubular tissue portion, the first member comprising a head, a base extending from the head, a plurality of threads extending from a surface of the base, and an aperture extending through the base and the head;
(b) placing a second member adjacent to the second tissue portion, the second member having an aperture therethrough and a plurality of threads extending from a surface of the aperture; and
(c) urging the base of the first member into the aperture of the second member in discrete intervals wherein the threads of the first member cooperate with the threads of the second member to control the distance that the base passes through the aperture of the second member.

21. The method of claim 20 further comprising cutting away a portion of the base that extends below the second member.

22. A surgical instrument for joining two tissue portions, the instrument comprising:
a handle;
a body extending from the handle; and
a head portion extending from the body, the head portion having an aperture, and a clip extending through the aperture, the clip being capable of grabbing a first end of a cable,
wherein a second end of the cable is fastened to a first member of a tissue joining device and the clip is adapted to translate the first member toward a second member of the tissue joining device to thereby fasten the first and second members together.

23. The surgical instrument of claim 22, further comprising a knob disposed proximate to the handle, wherein the knob is in communication with the clip, such that rotation of the knob translates the first member toward the second member.

24. The surgical instrument of claim 22, wherein the head portion includes a recess and the second member is positioned in the recess.

25. The surgical instrument of claim 22, wherein the clip comprises a cutting feature, wherein the cutting feature is adapted to penetrate a tissue portion.

26. The surgical instrument of claim 22, wherein the body is made of rubber.

27. The surgical instrument of claim 22 wherein the body comprises a plurality of ball and socket joints.

28. The surgical instrument of claim 22, further comprising a button for indicating the amount of force and thus displacement of the first member as it is tightened into the second member.

29. The surgical instrument of claim 28, wherein the button is located on the handle.

30. The surgical instrument of claim 22, wherein (i) the clip includes a first member and a second member attached to the first member, (ii) the first member of the clip includes a hook portion, and (iii) the second member of the clip is adapted to open and close an opening of the hook portion.

31. The surgical instrument of claim 30, wherein the first end of the cable includes a loop and the loop is capable of being grabbed by the clip when the second member of the clip is in an open position.

32. The surgical instrument of claim 22, further comprising a cutting mechanism disposed within the head portion wherein the cutting mechanism is adapted to cut away a portion of the first member of the tissue joining device that extends below the second member of the tissue joining device.

33. The surgical instrument of claim 32, further comprising a trigger coupled to the handle, and a cable coupled to the trigger at a first end and to the cutting mechanism at a second end, wherein when the trigger is squeezed the cutting mechanism cuts the portion of the first member that extends below the second member.

34. The surgical instrument of claim 32, wherein the cutting mechanism comprises a first blade.

35. The surgical instrument of claim 34, wherein the cutting mechanism comprises a second blade.

36. The surgical instrument of claim 22, wherein the head portion is detachable from the body.

37. The surgical instrument of claim 36, wherein the detachable head portion is compatible with an endoscope.

38. A method for joining two tissue portions, the method comprising:
(a) inserting a first member of a tissue joining device into a first lumen structure, a cord being attached to the first member;
(b) inserting a surgical instrument into a second lumen structure, the surgical instrument comprising a head portion having a clip;
(c) passing the clip through a wall of the second lumen structure;
(d) grabbing the cord with the clip;
(e) translating the first member to securely join the first member with a second member of the tissue joining device.

39. The method of claim 38, further comprising cutting a portion of the first member that extends below the second member.

40. The method of claim 38 further comprising suturing the first member to the first lumen structure.

41. The method of claim 38 further comprising rotating the first member when a base of the first member is positioned within an aperture of the second member.

42. The method of claim 38 further comprising rotating the second member when a base of the first member is positioned within an aperture of the second member.

43. The method of claim 38, wherein the second member is disposed within a recess formed in the head portion of the surgical instrument.

44. The method of claim 38, further comprising penetrating the wall of the second lumen structure with a cutting feature that extends from the clip, prior to step (b).

45. The method of claim 38, wherein the first member comprises a cutting feature, and as the first member is translated the cutting feature cuts the second lumen structure.

46. The method of claim 38, wherein the surgical instrument further comprises an endoscope.

47. The method of claim 38, further comprising guiding the clip into the head portion of the surgical instrument.

48. The method of claim 47, wherein the surgical instrument further comprises a visualization instrument.
